SEQUENCE LISTING (1 sequence)

SEQ ID NO: 1 - protein, 15 aa, Artificial Sequence
  Description of Artificial Sequence Synthetic peptide binding sequence
GGGGSGGGGSGGGGS

(19) United States
(12) Patent Application Publication
(10) Pub. No.: US 2005/0042718 A1
(43) Pub. Date: Feb. 24, 2005

(54) Method for preparing and selecting antibodies

(75) Inventors: Herve Bazin, Sceaux (FR); Yannick Nizet, Lincent Racour (BE)

Correspondence Address: IP DEPARTMENT OF PIPER RUDNICK LLP, ONE LIBERTY PLACE, SUITE 4900, 1650 MARKET ST, PHILADELPHIA, PA, 19103, US

(73) Assignee: Technopharm, a corporation of France, Paris (FR)

(21) Appl. No.: 10/674,130

(22) Filed: Sep. 29, 2003

Related U.S. Application Data
(63) Continuation of application No. PCT/FR02/01164, filed on Apr. 3, 2002.

(30) Foreign Application Priority Data
Apr. 3, 2001 (FR) 01/04525

(51) Int. Cl. C12P 21/02; C12N 5/06; C07K 16/18; C07H 21/04

(52) U.S. Cl. 435/69.1; 435/320.1; 435/326; 530/387.1; 536/23.53

(57) ABSTRACT

A method for preparing antibodies including a) transfecting a cell line with a nucleic acid construction including in the same reading frame a nucleic sequence coding for a membrane protein and a nucleic sequence of interest coding for a polypeptide of interest, and b) preparing antibodies directed against the polypeptide of interest with cells prepared in step (a) or with their membranes.

### RELATED APPLICATION

This is a continuation of International Application No. PCT/FR02/01164, with an international filing date of Apr. 3, 2002, now WO 02/081523 with a publishing date of Oct. 17, 2002, which is based on French Patent Application No. 01/04525, filed Apr. 3, 2001.

### FIELD OF THE INVENTION

This invention relates to an antigen presentation method for the preparation and, advantageously, the selection of antibodies, especially monoclonal antibodies.

### BACKGROUND

Monoclonal antibodies produced by hybridomas are of interest from multiple points of view and have been described exhaustively in many publications (Bazin, “Rat hybridomas and rat monoclonal antibodies”, CRC Press, 1990, 515 pages; Goding, “Monoclonal antibodies: principles and practice”, 3^{rd} edition, Academic Press, 1996, 492 pages; Shepherd and Dean, “Monoclonal antibodies”, Oxford University Press, 2000, 479 pages). These antibodies are useful in diagnostic as well as preventive and/or curative therapeutic application. This invention pertains more broadly to both in vivo as well as in vitro immunization.

Immune responses are directed against a substance which can be either a natural or artificial molecule with one or more epitopes or one or more haptens coupled to at least one carrier molecule. An adjuvant can be added to the antigen preparation. This antigen preparation, which will also be referred to below as “antigen”, corresponds to the classic definition in immunology manuals. The immune responses taken into consideration in the framework of this invention pertain to those described in immunology manuals and, in particular, those responses leading to the synthesis of antibodies.

Monoclonal antibodies directed against an antigenic determinant have been produced by a large number of laboratories since the introduction of the technique of cellular fusion between a myeloma cell and a lymphoid cell of an immunized animal. The first model of hybridomas secreting monoclonal antibodies developed by Köhler and Milstein (1975, Nature, vol. 256, page 495) was a murine model. This was extended to the rat (Galfré et al., 1979, 277, 131) and is widely used.

However, in addition to the fusion technique, the production of a monoclonal antibody depends above all on the immunization method employed and the correct screening of the hybrid clones of interest. There is a broad distinction between two types of immunization of animal organisms, either in vivo or in vitro, and selecting certain of their cells and culturing them with an antigen.

A further distinction is made of three immunization techniques: i) immunization with the purified antigen of interest, ii) immunization with a cell having the antigen of interest, iii) DNA immunization consisting of administering a DNA sequence (plasmid) expressing the gene coding for the antigen of interest. In all three cases, it is known that the immune reaction leading to the production of antibodies directed against the antigen requires simultaneous activation of the B lymphocytes, specifically recognizing the native antigen and, most often, the T lymphocytes specifically recognizing the degraded antigen and, presented in peptide form by the molecules of the major histocompatibility complex of cells referred to as antigen presenters.

Conventional immunization under these various modalities has been described for almost a century, whereas in-vitro immunizations are more recent. An example includes the techniques described by Mishell and Dutton in the mouse or that described by various groups in humans.

Immunization by a protein requires purification in advance of the antigen or its production in a recombinant form which does not always have the conformational and post-transcriptional characteristics of the native protein.

A disadvantage of immunization with a cell is the large diversity of the antibodies generated which recognize not only the protein of interest but also the multitude of other antigens in the cells, which makes it difficult to characterize the monoclonal antibodies obtained.

Finally, immunization by the so-called “naked DNA” methods has already been employed successfully (Costaglioga et al., Journal of Immunology 1998, 160, 1458-1465), but remains limited at the level of the magnitude of the immunological responses obtained. In fact, these methods require that the gene coding for the antigenic protein be expressed in the host cells of the immunized subject and that this protein be then presented on the surface of the host cells or secreted ,by these cells. Moreover, these methods do not allow screening of the clones obtained.

These data are applicable both for the classic immunization of an animal like the rat, mouse or any other species capable of yielding humoral immune or cellular responses, which might be normal or modified by transgenic techniques. They are valid for in-vivo as well as in-vitro immunizations.

It Was possible to obtain monoclonal antibodies in the mouse by immunizing it with non-immune autologous or allogenic mouse lines that were transfected and expressed the antigen of interest on their membranes (Palmer D., Kevany M., Mackworth-Young C., Batchelor R., Lombardi G., Lechler R. “Generation and characterization of an HLA-DR specific monoclonal antibody using L-cell transfectants expressing human and mouse class II major histocompatibility dimers. Immunogenetics 33(1): 12-17, 1991. Thurau S. R., Wildner G., Kuon W., Weiss E. H., Rithumuller G. Expression and immunogenicity of HLA-B27 in high transfection recipient P815: a new method to induce monoclonal antibodies directed against HLA-B27. Tissue Antigen 33(5): 511-519, 1989).

It would therefore be advantageous to provide a method of immunization which makes it possible to easily obtain very specific immunologic responses and easily screen the antibodies formed.

### SUMMARY OF THE INVENTION

This invention relates to a method for preparing antibodies including a) transfecting a cell line with a nucleic acid construction including in the same reading frame a nucleic sequence coding for a membrane protein and a nucleic sequence of interest coding for a polypeptide of interest, and b) preparing antibodies directed against the polypeptide of interest with cells prepared in step (a) or with their membranes.

### DETAILED DESCRIPTION

This invention in one aspect relates to a method for the preparation of antibodies comprising:
a) transfection of a cell line by a nucleic acid construction comprising a nucleic sequence of interest coding for a polypeptide of interest to be expressed at surfaces of cells of the -cell line, and
b) preparation of antibodies directed against the polypeptide of interest With the cells prepared in step (a) or with their membranes.

In the framework of the invention, the term “membrane” refers without distinction to intact cell membranes and their fragments obtained by techniques known in the art. The cells can be immortal lines as well as cells with an extended lifetime such as, for example, fibroblasts. According to a preferred embodiment, the cell line employed in transfection step (a) is a cell line of the immune system, for example, a lymphoid line.

The choice of these immune system cells is determinant because they are capable of naturally colonizing the lymphoid organs, in which is produced the immune reaction leading to the production of antibodies. Moreover, this transfected line used for immunization can serve as well as line for fusion with the B lymphocyte that produces and expresses at its surface the antibodies directed against the antigen expressed by the transfected line. Thus, in accordance with the method of the invention, one obtains advantageously a link between the two types of cells which increases the yield of the fusion.

The prepared antibodies are then selected in step (b) by any immunization or molecular biology method known in the art. Step (b) is advantageously implemented:
(i) by immunization of an animal with transfected cells expressing at their surface the polypeptide of interest prepared in step (a) or with their membranes, and
(ii) by bringing into contact transfected cells expressing at their surface the polypeptide of interest prepared in step (a) or their membranes with cell populations comprising cells capable of producing antibodies.

The term “cells capable of producing antibodies” should be understood to mean any cell effectively producing antibodies, as well as any cell possessing the equipment necessary for their assembly and production, but not producing them, for example, because of the simple fact of its immature stage of development.

The invention pertains most particularly to a method for the preparation of antibodies in which the cell line transfected in step (a) is histocompatible with the animal immunized in step (i) or the cells employed in step (ii).

The method of the invention can be implemented with the cells of any animal capable of producing antibodies such as, of course, mammals. An immune system line is preferably employed, for example, a lymphoid line. The transfected cell line can be any animal line, including mammals and, more particularly, humans. A particular example includes a rodent myeloma line, preferably a rat myeloma line and, most particularly, a rat myeloma line of strain LOU such as the rat myeloma line LOU IR 983 F/TEC deposited with the National Collection of Microorganism Cultures of Institut Pasteur (Paris) as No. I-2584 on Nov. 29, 2000.

In a preferred aspect, the transfection of a cell line in step (a) is performed with a nucleic acid construction comprising a nucleic sequence of interest coding for a membranal polypeptide of interest. The nucleic sequence of interest is, of course, placed under the control of regulation sequences which enable the elevated expression of the polypeptide of interest and its exportation to the surface of the transfected cells.

According to another aspect, the transfection of a cell line in step (a) is performed with a nucleic acid construction comprising in the same reading frame a nucleic sequence coding for a membrane protein called “auxiliary membrane” and a nucleic sequence of interest coding for a polypeptide of interest. The nucleic sequence coding for an auxiliary membrane protein and the nucleic sequence of interest are, of course, placed together under the control of regulation sequences enabling the elevated expression of the polypeptide of interest and its exportation to the surface of the transfected cells. The nucleic sequence of interest can be placed before, after or in the nucleic sequence coding for an auxiliary membrane protein. In this form of implementation, the nucleic sequence coding for an auxiliary membrane protein and the nucleic sequence coding for a polypeptide of interest can be directly bound to each other or bound by one or more identical or different binding nucleic sequences. These can be binding sequences coding for a relatively inert peptide or polypeptide whose purpose is solely to prevent interactions between the auxiliary membrane protein and the polypeptide of interest. The binding nucleic sequence can also code for a polypeptide participating in the immune response. As an example of such a binding sequence, we can cite the following sequence: GGGGSGGGGSGGGGS.

The nucleic acid construction employed for transfecting the cell line of step (a) advantageously comprises a selection gene, for example, a resistance gene to an antibiotic such as neomycin.

The nucleic acid construction employed for transfecting the cell line of step (a) is preferably a nucleic vector adapted to the cells of the line transfected in step (a).

One particular example of a vector according to the invention comprises from 5′ to 3′:
a promoter such as the promoter Srα,
a leader sequence such as that of mouse CD80,
a cloning polysite in which is inserted the gene of the polypeptide of interest,
a binding nucleotide sequence,
the nucleotide sequence coding for a membrane polypeptide such as mouse CD80, possibly preceded by a leader sequence such as the mouse CD80 leader sequence.

In an especially preferred aspect, the cell line transfected in step (a) is autologous, isologous or homologous with the cells employed in step (i) or (ii) for the preparation of antibodies.

After transfection of the cell line in step (a), the transfectants are advantageously analyzed before step (b) for expression of the auxiliary membrane protein or the polypeptide of interest.

Thus, after transfection of the cell line with the vector and selection in a medium corresponding to the resistance gene, the transfectants are analyzed by flow cytometry for the expression of the auxiliary membrane protein or the polypeptide of interest, for example, by means of a monoclonal antibody directed against one of these polypeptides. The selected clones have the polypeptide of interest at their surface.

The antibodies can be polyclonal or monoclonal depending on the technique employed in step (b). Thus, a first form of implementing step (b) comprises preparing monoclonal antibodies directed against the polypeptide of interest. This mode of implementation comprises a technique based on cellular fusion from animal cells having received transfected cells expressing at their surface the polypeptide of interest prepared in step (a) or their membranes. These cells or their membranes are advantageously administered to the animal alone or in combination with an adjuvant. The administration can be implemented once or multiple times. It can be useful to irradiate the cells prior to immunization to diminish or block their division mechanism. After immunization and fusion of the cells by the previously described techniques, the clones producing the monoclonal antibodies directed against the polypeptide of interest can be detected, for example, by flow cytometry comparing their attachments on the cells expressing the surface protein but transfected by the vector not comprising the polypeptide of interest (“empty” vector) and the cells expressing the surface protein transfected by the vector also comprising the polypeptide of interest.

The method is of particular value in association with the technique of Köhler and Milstein for the preparation of hybridomas synthesizing monoclonal antibodies with the various adaptations of the method to the mouse, the rat or other species, but also with methods employing an in-vivo immunization (with an optionally transgenic animal) or an in-vitro immunization. The employment of completely histocompatible systems is highly preferred, but its use is not excluded in a non-histocompatible system in which the values:
of the immunization system would be partially conserved because of the identity of the majority of the antigens between the system to be immunized and the immunizing system,
of the also practically conserved screening except for alloantigens or xenoantigens.

Thus, a second form of implementation of step (b) of the method of the invention consists of bringing into contact transfected cells, for example, IR983F or Sp₂/O cells sensitive to the HAT medium, expressing on their surface the polypeptide of interest, prepared in step (a), or their membranes, with cells producing antibodies. It is thereby possible to immortalize one or multiple B lymphocytes stemming from this immune response and to obtain a rapid and easy screening of the clones originating from the various techniques employed, by means of the classic cell fusion according to Köhler and Milstein, but also by means of transgenic animals synthesizing, for example, human antibodies and, in fact, by means of any system producing specific antibodies from immature or memory B lymphocytes, stimulated by means of antigen, in an in-vivo culture (for example, by transfer of human cells into immunodeficient animals) or an in-vitro culture. In the case of B lymphocytes already producing specific targeted antibodies (activated B lymphocytes, preplasmacytes, plasmacytes, etc.), the method described below conserves its value at the level of screening antibodies coming from immortalized cells.

A third form of implementation of step (b) comprises:
isolation from B cells producing antibodies against the polypeptide of interest, of nucleic acid sequences coding for each of the chains of said antibodies,
expression in a host of the nucleic acid sequences.

The method can comprise after step (b) a capacity test of the antibodies obtained to recognize the polypeptide of interest by bringing the antibodies into contact with:
the cells transfected in step (a) and/or
cells transfected with a nucleic acid construction identical to that used in step (a), but without the nucleic sequence of interest.

The method is remarkable in that it makes it possible to immortalize cells from the same organism, for example, the same individual of the human species who could have certain of B lymphocytes immortalized by the very classic Epstein-Barr virus technique and other cells collected in the same blood sampling and conserved by freezing or another subsequent time or collected from any other tissue enabling collection of cells from the same individual in a sufficient quantity. In addition to immortal cells, the method can be implemented with cells with a long duration of life such as, for example, fibroblasts.

By selecting the appropriate cell line and expression system, the method enables expression of the antigen in cells having not only class I histocompatibility with the cells to be immunized but, moreover, of choosing cells expressing class II histocompatibility molecules. In this latter case, the transfected cells can present T epitopes of the antigen, if it possesses them, to the T CD4 lymphocytes of the immunization system.

The method is also remarkable in that it does not require knowledge of nor advance preparation of the polypeptide sequence or even the entire molecule on which the targeted antibodies must be able to bind specifically. In fact, when the antigen coded by the adequate construction is presented to the immunization system (adequate animal or cell preparation), the antigen alone is considered to be foreign (the not-itself of classic immunology manuals) in the in-vivo or in-vitro system, the great majority of the antibodies would be practically directed against it. There are various theories which differ on the point of knowing whether all or a fraction of the antibodies formed during an immune response are directed against the antigen introduced into the system. In practice, an immune response is considered to be specific when the large majority of the antibodies is directed against the antigen employed. In this case, the antibodies can be directed against the induced antigens as well as by the nucleic acid construction. The drawbacks associated with this problem can be avoided by obtaining adequate control cells by means of an adequate transfection, all having the antigens of the transfected cells with the exception of the antigen being studied.

The method is also remarkable in that it makes it possible to considerably simplify the step of screening the antibodies of interest. It is sufficient to compare the attachment of antibodies from a supernatant of a culture to be tested on the cells of the original cell line or the line transfected by the same nucleic acid construction with the exception of those coding for the antigen, to those of the transfected line with the nucleic acid construction incorporating those coding for the antigen. This type of comparison can be made using a type of device called a FACS (Fluorescent Antibody Cell Sorter). The use of a second antibody tagged with fluorescein and selected such as to be capable of attaching to the first can enable an easy detection of the first antibody whose presence is targeted. This type of technique is of common usage in specialized laboratories. Other methods providing a difference between the control cells and the transfected cells can be employed.

The method can be applied in various fields requiring the development of monoclonal or even polyclonal antibodies, which can be employed in research for diagnostics or therapeutics. From a direct therapeutic point of view, the method can be used for the immunization of patients in which the strict control of the fate of the transfected cells which are inoculated in the patient must be ensured. Thus, for example, the method can be used for inducing an immune response in a patient by using a transfected autologous line that has been treated to prevent its division, or also using membrane fragments of the cells of that line.

The invention also pertains to a nucleic acid construction such as defined above and capable of being employed in the method. The invention also pertains to cells obtained in step (a) of the method and the compositions containing them, and also to compositions comprising their membranes as well as compositions comprising an antibody obtained by the method. The invention finally pertains to a treatment or diagnostic method applied to a subject consisting of implementing the method of the invention in vivo with a polynucleotide sequence coding a protein of therapeutic or diagnostic interest.

Other advantages and characteristics of the invention will become apparent from the examples presented below as nonlimitative examples of implementation of the method of the invention.

### EXAMPLE I

The nonsecretory myeloma line IR 983 F, which is histocompatible with LOU/C rats and their first-generation hybrids such as the rats LOU/C X OKAMOTO and LOU/C X PVG/c, was transfected by electroporation by means of the plasmid pBJ LL177 (Azuma M, Cayabyab M, Buck D, Phillips J H and Lanier L L. CD28 interaction with B7 costimulates primary allogenic proliferative response and cytotoxicity mediated by small, resting T lymphocytes. J. Exp. Med. 175: 353-360, 1992).

This plasmid obtained from ATCC (ATCC number 99595) codes for human CD80 under the control of the promoter SRα (Takabe Y, Seiki M, Fujisawa J F, Hoy P, Yokota K, Arai K I, Yoshida M and Arai N. Srα promoter: an efficient and versatile mammalian cDNA expression system composed of the simian virus 40 early promoter and the R-U5 segment of human T-cell leukemia virus type 1 long terminal repeat. Mol. Cell. Biol. 8: 466-472, 1988). This plasmid also presents a neomycin resistance gene.

Electroporation conditions: 5 million cells in 500 μl of medium presenting the plasmid at the concentration of 10 μg/ml received a pulse of 300 volts.

After selection in a medium containing neomycin (1 mg/ml), the transfectants were analyzed by flow cytometry for the expression of human CD80 using the monoclonal antibody anti-CD80 BB1 (Pharmingen). The clone expressing at its surface the most human CD80 was developed.

An LOU/C rat received two intraperitoneal injections, spaced apart by two months, of positive IR983F CD80 (2×10⁷ per injection), irradiated at 2.5 Gy with a cesium source, emulsified in Freund's complete adjuvant for the first injection and in Freund's incomplete adjuvant for the second injection. Two weeks after the second immunization, the serum of the immunized animal presented antibodies recognizing IR983F expressing human CD80 but not recognizing normal IR983F.

An LOU/C rat also received two intraplantar injections, spaced apart by two weeks, of positive IR983F CD80 (10⁷ cells per paw), irradiated with cesium at the dose of 2.5 Gy, emulsified in Freund's complete adjuvant for the first injection and in Freund's incomplete adjuvant for the second injection. Four days after the second immunization, the popliteal ganglia were collected and the cells were fused with IR983F.

Seventeen hybridomas producing a monoclonal antibody recognizing IR983F expressing human CD80 and not recognizing non-transfected IR983F were obtained. Three were isotype IgG1, 9 isotype IgG2a, 4 isotype IgG2b and one isotype IGM.

These antibodies also recognized the human B line DAUDI which also expresses CD80.

The method described above enables production of antibodies directed solely against a membranal protein. An expression vector enabling expression of polypeptide at the surface of IR983F was prepared to generalize the method to all proteins. This vector comprised respectively from 5′ to 3′:
the promoter Srα,
the leader sequence of mouse CD80,
a cloning site Sfi I/Not I enabling insertion of the gene coding for a polypeptide of interest,
a nucleic sequence coding for an auxiliary polypeptide binding chain having as its motif: GGGGSGGGGSGGGGS, and
the coding part of mouse CD80 with the exception of the leader sequence. The vector also had available the neomycin resistance gene.

After transfection of IR983F with this vector and selection in a medium containing neomycin, the transfectants were analyzed by flow cytometry for the expression of mouse CD80 by means of a monoclonal mouse anti-CD80 antibody.

The clones which expressed mouse CD80 necessarily expressed at their surface the following polypeptide sequence:

MEMBRANE-mouseCD80-GGGGSGGGGSGGGGS-POLYPEPTIDEOFINTEREST-NH2.

After immunization and fusion of the cells, the clones producing monoclonal antibodies directed against the polypeptide of interest were detected by flow cytometry by comparing their attachments on the IR983F transfected by the “empty” vector in which the sequence of interest was not cloned and which thus did not express mouse CD80, and the IR983F transfected by the vector comprising the sequence of interest cloned in fusion with the mouse CD80.

### EXAMPLE II

The nucleic sequence coding for the first 250 amino acids of the protective antigen of Bacillus anthracis (Sequence and analysis of the DNA encoding protective antigen of Bacillus anthracis. Welkos S L, Lowe J R, Eden-McCutchan F, Vodkin M, Lppla S H, Schmitt J J. Gene 69: 287, 1988) was cloned in the expression vector described above after Sfil/NotI restriction.

IR983F [cells] were transformed either with the “empty” expression vector or with the expression vector containing the Bacillus anthracis gene.

After selection in a medium containing neomycin (1 mg/ml), the transfectants expressing mouse CD80, detected by flow cytometry using the monoclonal antibody MCA 1586F (Serotec), were obtained.

The clones expressing the most mouse CD80 at their surface were developed.

These transfectants were then used for immunization of animals.

For example, an LOU/C rat received two intraplantar injections, spaced apart by two Weeks, of positive IR983F-CD80 transfected by the expression vector comprising the Bacillus anthracis gene (10⁷ cells per paw), emulsified in Freund's complete adjuvant for the first injection and in Freund's incomplete adjuvant for the second paw. Four days after the second immunization, the popliteal ganglia were collected and the cells fused with IR983F.

This resulted in hybridomas producing a monoclonal antibody recognizing positive IR983F-CD80 transfected by the vector comprising the Bacillus anthracis gene, but not recognizing the positive IR983F-CD80 solely transfected by the “empty” vector.

### EXAMPLE III

The non-secretory myeloma line IR983F was transfected by electroporation using the plasmid pBJ CD94. This plasmid was constructed by replacing the cloned CD28 gene in the plasmid PBJ LL177. This plasmid also had a neomycin resistance gene.

Electroporation conditions: 5 million cells in 500 μl of medium having the plasmid at a concentration of 10 μg/ml received a pulse of 300 volts.

After selection in a medium containing neomycin (1 mg/ml), the transfectants were analyzed by flow cytometry for expression of human CD94 using the HP-3D9 anti-CD94 monoclonal antibody (Pharmingen). The clone expressing the most human CD94 at its surface was developed.

An LOU/C rat received two intraperitoneal injections, spaced apart by two months, of positive IR 983F CD94 (2×10⁷ per injection), irradiated at 2.5 Gy with a cesium source, emulsified in Freund's complete adjuvant for the first injection and in Freund's incomplete adjuvant for the second injection. Two weeks after the second immunization, the serum of the immunized animal had antibodies recognizing IR983F expressing human CD94, but not recognizing normal IR983F.

An LOU/C rat also received two intraplantar injections, spaced apart by two weeks, of positive IR 983F CD94 (10⁷ cells per paw), irradiated by cesium rays at the dose of 2.5 Gy, emulsified in Freund's complete adjuvant for the first injection and in Freund's incomplete adjuvant for the second injection. Four days after the second immunization, the popliteal ganglia were collected and the cells fused with IR983F or IR983F expressing human CD94.

Eight hybridomas producing a human anti-CD 94 monoclonal antibody and not recognizing non-transfected IR983F were obtained by fusing 26 million ganglial cells with IR983F. Seventeen hybridomas producing a human anti-CD94 antibody were obtained by fusing 20 million ganglial cells with IR983F expressing human CD94.

All of the antibodies recognized both IR983F CD94 and the human NK that expressed CD94.

The isotypes of these clones were: 3 IgM, IgG1, 15 IgG2a, 5 IgG2b, IgG2c.

This experiment was repeated using IR983F expressing human ligand CD134 as the immunization line. Four hybridomas producing an anti-CD134L antibody were obtained by fusing 30 million ganglial cells with IR983F. Ten hybridomas producing an anti-CD134L antibody were obtained by fusing 30 million cells expressing human CD134L. The antibodies recognized both IR983F CD 134L and the human B lymphocytes that express CD134L.

### EXAMPLE IV

IR983F membranes were obtained, for example, by Dounce homogenization (Koizumi K, Shimizu K T, Nishida K, Sato C, Ota K, Yamamada N. Biochim-Biophys. Acta 649: 393-403, 1981).

An LOU/C rat received three subcutaneous injections, spaced apart by two days, of membranes corresponding to 40 million IR983F cells expressing human CD70.

Ten days after the first injection, polyclonal antibodies recognizing specifically IR983F cells expressing human CD70 were detected by flow cytometry in the serum of the immunized animal.

### EXAMPLE V

The method of the invention can be implemented with SP₂/O cells as cell line capable of being transfected in step (a) of the method.

This particular implementation example is described below.

Material

Complete RPMI medium
a 500-ml flask of RPMI
−5 ml of L-glutamine
−500 [μl of gentamicin (at 50 mg/ml)
   Electroporation medium (qsp 300 ml, sterile distilled water)
−0.3 M inositol (16.2 g)
+1 mM KH₂PO₄ (40.82 mg)
+0.1 mM calcium acetate (4.74 mg)
+0.5 mM magnesium acetate (32.17 mg)
   Post-electroporation medium (qsp 300 ml, sterile distilled water)
132 mM NaCl (2.31 g)
+8 mM KCl(178.9 g)
+10 mM KH₂PO4 (408 mg)
+0.1 mM calcium acetate (4.74 mg)
+0.5 mM magnesium acetate (32.17 mg)
   Method
SP₂/O cell counting was performed from the culture flasks.
−1·10⁷ cells were collected.
The collected cells were centrifuged for 5 minutes at 1300 rpm.
The residue was resuspended in a milliliter of electroporation buffer at 37° C.
−10 μg of plasmid CD40L was added. (The PBJ-CD40L plasmid was constructed by replacing the cloned CD28 gene in plasmid PBJ LL177 with that of CD40L initially cloned in BCMGSneo-TRAP (Cloning of TRAP, a ligand for CD40 on human T-cells. Eur. J. Immunol. 22: 3191-3194, 1992) or plasmid CD94, depending on the case.
The resultant suspension was homogenized by compression aspiration.
−400 μl of the suspension was collected.
The cell suspension in the presence of the plasmid was subjected to electroporation in conical 400-μl tubes under the following conditions: 350 V, 5 ms, one pulse.
One milliliter of post-electroporation medium at 37° C. was then added to the tube.
The suspension was incubated for 10 minutes at ambient temperature.
The content of the tube was then collected and the cell suspension was resuspended in RPMI medium without phenol red (Invitrogen).

The cells were then distributed at the rate of 200 μl per well in a 96-well culture plate.

They were incubated for 24 h at 37° C. under a 5% CO₂ atmosphere.

The changing of the culture medium was performed by addition of 150 μl per well of complete RPMI medium (Invitrogen) containing 0.5 mg/ml of geneticin (Invitrogen).

The cells were incubated again at 37° C. under a 5% CO₂ atmosphere.

The cells transfected by the plasmid were allowed to develop (resistance to geneticin).

FACS analysis of the cells transfected by the plasmid was performed to detect the membranal expression of CD40L or CD94, depending on the case.

The following were obtained from the experiments performed according to the method described above:
One transfected SP₂/O clone expressing CD40L (SP20-CD40L),
Three SP2/O clones expressing CD94 (SP₂/O—CD94-F4, SP/O—F1 and SP₂/O—CD94-D2).

1. A method for preparing antibodies comprising:
a) transfecting a cell line with a nucleic acid construction comprising in the same reading frame a nucleic sequence coding for a membrane protein and a nucleic sequence of interest coding for a polypeptide of interest, and
b) preparing antibodies directed against the polypeptide of interest with cells prepared in step (a) or with their membranes.

2. The method according to claim 1, wherein the cell line transfected in step (a) is an immune system line.

3. The method according to claim 2, wherein the cell line transfected in step (a) is a fusion line with a B lymphocyte.

4. The method according to claim 1, wherein preparation of antibodies in step (b) is performed from B cells obtained:
(i) by immunizing an animal with transfected cells expressing at their surface the polypeptide of interest prepared in step (a) or with their membranes,
(ii) by contacting transfected cells expressing at their surface the polypeptide of interest prepared in step (a) or their membranes with cell populations comprising cells capable of producing antibodies.

5. The method according to claim 4, wherein the cell line transfected in step (a) is histocompatible with the animal immunized in (i) or the cells employed in (ii).

6. The method according to claim 1, wherein the nucleic sequence of interest is placed under control of regulation sequences enabling elevated expression of the polypeptide of interest and its exportation to surfaces of the transfected cells.

7. The method according to claim 1, wherein the nucleic sequence coding for a membrane protein and the nucleic sequence of interest are placed together under control of regulation sequences enabling elevated expression of the polypeptide of interest and its exportation to surfaces of the transfected cells.

8. The method according to claim 1, wherein the nucleic sequence of interest is placed before, after, or in the nucleic sequence coding for a membrane protein.

9. The method according to claim 1, wherein the nucleic sequence coding for a membrane protein and the nucleic sequence coding for a polypeptide of interest are bound by one or more identical or different binding nucleic sequences.

10. The method according to claim 9, wherein one of the binding nucleic sequences codes for a polypeptide participating in the immune response.

11. The method according to claim 1, wherein the nucleic acid construction employed for transfecting the cell line of step (a) comprises an antibiotic resistance gene.

12. The method according to claim 1, wherein the nucleic acid construction employed for transfecting the cell line of step (a) is a nucleic vector adapted to the cell line transfected in step (a).

13. The method according to claim 1, wherein the transfected cell line is autologous, isologous or homologous with the cells employed in (i) or (ii).

14. The method according to claim 1, further comprising, after transfection of the cell line, analyzing the transfectants for expression of the membrane protein or polypeptide of interest.

15. The method according to claim 1, wherein in step (b), the monoclonal antibodies are prepared by cellular fusion from cells of an animal having received transfected cells expressing at their surface the polypeptide of interest prepared in step (a) or their membranes.

16. The method according to claim 1, wherein step (b) comprises:
isolation from B cells producing antibodies against the polypeptide of interest, of nucleic acid sequences coding for each of the chains of said antibodies, and
expressing the nucleic acid sequences in a host.

17. The method according to claim 1, further comprising, after step (b), testing the antibodies obtained for capacity to recognize the polypeptide of interest by contacting the antibodies with:
the cells of the line transfected in step (a) and/or the cells of the line transfected with a nucleic acid construction identical to that used in step (a), but without the nucleic sequence of interest.

18. A cell into which has been introduced a nucleic acid construction comprising in the same reading frame a nucleic sequence coding for a membrane protein and a nucleic sequence of interest coding for a polypeptide of interest.

19. A composition comprising cells as defined in claim 18 or membranes of the cells.

20. The method of claim 2, wherein the immune system line is a lymphoid line.
